(19) United States
(12) Patent Application Publication
(10) Pub. No.: US 2015/0327765 A1
(43) Pub. Date: Nov. 19, 2015

(54) APPARATUS AND METHOD FOR IMAGING VASCULATURE AND SUB-DERMAL STRUCTURES BY TRANS-ILLUMINATING NIR LIGHT

(71) Applicant: INFRARED IMAGING SYSTEMS, INC., Columbus, OH (US)

(72) Inventors: Robert L. CRANE, Dayton, OH (US); Steven MERSCH, Germantown, OH (US); James W. SHARPE, Radnor, PA (US); Dale SIEGEL, Marysville, OH (US)

(73) Assignee: INFRARED IMAGING SYSTEMS, INC., Columbus, OH (US)

(21) Appl. No.: 14/367,290

(22) PCT Filed: Dec. 21, 2012
(86) PCT No.: PCT/US2012/071397

§ 371 Date: Jun. 20, 2014

Related U.S. Application Data
(60) Provisional application No. 61/579,035, filed on Dec. 22, 2011.

(51) Int. Cl. A61B 5/00; H04N 5/225; H04N 5/232; H04N 5/235; H04N 7/18; H04N 5/33

(52) U.S. Cl. CPC A61B 5/0059; H04N 7/185; H04N 5/33; H04N 5/23296; H04N 5/23293; H04N 5/2354; H04N 5/2251; A61B 5/7435; A61B 5/002; A61B 2019/5231

(57) ABSTRACT

A system for real-time visualization of subdermal structures of a mammal, using near-infrared (nIR) illumination source, a support structure with independently articulating arms for attaching a camera and a visual display screen, a controller for the camera and nIR illumination source. The camera includes a zoom lens that provides a detection field of view at a long working distance to avoid the camera obstructing the view of the medical personnel when performing a medical procedure on the mammalian body part. A targeting system indicates a focus location of the zoom lens and a center of detection field of view. An nIR bandpass filter and image processor convert the captured and filtered trans-illuminating nIR light to an image signal. An interfaced computer can operate on a commercial or proprietary operating systems and operates image enhancement software and image archival, distribution and display.

### CROSS-REFERENCE TO RELATED APPLICATIONS

This application is a US National Stage of International Application No. PCT/US2012/071397, filed Dec. 21, 2012 (pending), which claims the benefit of U.S. provisional application 61/579,035, filed Dec. 22, 2011 (expired), the disclosures of which are incorporated herein by reference in their entirety.

### BACKGROUND OF THE INVENTION

Medical diagnosis, treatment and therapy methods and systems can employ the transmission and imaging of near-infrared light into and through the human body for viewing blood vessels and other sub-dermal structures in the body. The administration of medical care to a patient often requires vascular access. Expeditious administration of medical care to the victim or patient improves the prospects of recovery for the victim or patient. Patients may have veins that are partially collapsed, or veins that are difficult to find or difficult to access (such as in the treatment of infants or geriatric persons), which further complicates procedures for gaining access to the veins. The treatment of patients requiring vascular access may also be complicated by patient size (a neonate), obesity, skin pigmentation or other physical characteristic that can reduce peripheral circulation.

In the practice of the procedures for visualization of subcutaneous structures by trans-illumination using infrared or near-infrared light, proper support of the light source in order to effectively direct the light onto a body portion of interest may be an awkward procedure for the health care provider in treating a patient. U.S. Pat. No. 7,925,332, issued to Crane et al on Apr. 12, 2011 (the disclosure of which is incorporated herein by reference) discloses a multi-layered structure in the form of a disposable patch useful in conjunction with procedures for the non-invasive visualization of veins, arteries or other subcutaneous structures of the body or for facilitating vascular insertion of needles or catheters for administration of fluids and medication, measurement of physiological parameters, extraction of venous or arterial blood, or the like. The patch is particularly useful in conjunction with systems and methods for the detection and display of subcutaneous structures such as described in U.S. Pat. No. 6,230,046 to Crane et al (the disclosure of which is incorporated herein by reference), which describes systems and methods for enhancing the visualization of veins, arteries or other subcutaneous natural or foreign structures in the body and for facilitating vascular (both venous and arterial) insertion or extraction of fluids, medications or the like in the administration of medical treatment to a patient, including a light source of selected wavelength(s) for illuminating or trans-illuminating a selected portion of the body and a low-level light detector and suitable filters for generating an image of the illuminated body portion.

US Patent Publication US 2004-0215081 (the disclosure of which is incorporated herein by reference) discloses a real-time visualization and detection of an extravasated or infiltrated fluid in subdermal or intradermal tissues at a site of an intravascular injection by illuminating an intended site of an intravascular injection with infrared light from a light source and generating real-time images of the body and the injected fluid to determine differences in contrast evidencing extravasation or infiltration of said fluid near the vasculature within the body.

Medical technicians and professionals work under a variety of lighting conditions, including surgical operating rooms, clinics, and doctor's offices that employ high intensity lighting, fluorescent lighting, incandescent lighting, and visible LED lighting. Medical personnel can use different modes of visualizing the trans-illuminated IR light. In one type of procedure, the medical personnel can view the trans-illuminated infrared light employing an intensifier tube or similar display device similar to night vision goggles, as described in Crane et al, supra. In another procedure, an image of the trans-illuminated IR light is displayed on a display device or monitor that the medical personnel view with the unaided eyes. Such display device or monitor can be a liquid crystal display (LCD), LED display, gas plasma, cathode ray tube or other display that receives an image of the infrared light captured by a camera or imaging device. The display device can be within reach of the medical personnel as shown in PCT Patent Publication WO 2010/059045 (the disclosure of which is incorporated by reference in its entirety), or on a computer screen or display remote from the patient.

In ambient lighting that has an output having a cycled maxima and minima, such as fluorescent lights, the pulsing of the IR light source can be synchronized with the minima of the output from the ambient room, and gated with the light detector (camera), as described in US Patent Publication 2004-0215081, the disclosure of which is incorporated by reference in its entirety.

The work of medical personal is highly skilled and requires focus and attention to perform procedures and diagnose medical conditions with a minimum of distraction and complexity. Despite numerous advances in the illumination and trans-illumination of the human body with infrared light, in the detection of trans-illuminated infrared light from the illuminated body, and in the imaging and viewing of the detected light signals, there remains a need for improved methods and systems for use by medical personnel to provide high quality images of the sub-dermal structures that is convenient, rapidly deployable, and easy to use and avoids confusion and complexity.

### SUMMARY OF THE INVENTION

The present invention provides an imaging system for visualizing, including real-time visualization of, sub-surface structures, including sub-dermal structures, in a body part, including an extremity, of an animal, typically a mammal. The system includes a near-infrared (nIR) illumination source that emits nIR light that trans-illuminates the body part of the animal. The imaging system also includes a camera that captures the trans-illuminating nIR light. The camera typically includes a zoom lens to provide a detection field of view at a long working distance for the camera from the animal body part, the long working distance being sufficient to avoid the camera obstructing a visual field of view of a user, typically a medical personnel, when performing a procedure such as a medical or examination procedure on the body part. The camera can be attached to the distal end of the upper arm.

An imaging system can also include a targeting system for indicating a center of detection field of view, and optionally a focus distance of the zoom lens. The imaging system can also include an image processor for converting the captured trans-illuminating nIR light to an image signal. The imaging system also includes a visual display device, which can be attached to a distal end of the lower articulating arm. The visual display device can include a visual display screen, at least one controller for sending a control signal to the camera, for sending power and control signals to the nIR illumination source, and for transmitting the processed image signal to the visual display screen.

The invention also can provide an imaging system for visualization, typically in real time, of surface and sub-surface structures in a body part or an extremity of an animal, the system including: a near-infrared (nIR) illumination source that emits nIR light that trans-illuminates an animal body part; a camera including a zoom lens; a targeting system for indicating a focus location and a center of detection field of view of the zoom lens; an image processor for converting the captured trans-illuminating nIR light to an image signal; and a visual display device including a controller for sending a control signal to the camera, and for sending power and control signals to the nIR illumination source, and a display screen that receives and displays the processed image signal.

The present invention can provide an imaging system for real-time visualization of sub-surface structures in a body part of a mammal, the system including: a near-infrared (nIR) illumination source that emits nIR light that trans-illuminates the body part; a support structure that includes an upright post, a lower arm extending from the upright post, and an upper arm extending from an upper portion of the upright post and including a distal end, wherein the upper arm and the lower arm articulate independently; a camera attached to the distal end of the upper arm that captures the trans-illuminating nIR light, the camera including a zoom lens to provide a detection field of view at a long working distance for the camera from the body part, the long working distance being sufficient to avoid the camera obstructing a visual field of view of the medical personnel when performing a medical procedure on the body part; a targeting system associated with the camera for indicating a focus location of the zoom lens and a center of detection field of view; an image processor for converting the captured trans-illuminating nIR light to an image signal; a visual display device attached to a distal end of the lower articulating arm and including a visual display screen; and at least one controller for sending a control signal to the camera, for sending power and control signals to the nIR illumination source, and for transmitting the processed image signal to the visual display screen.

The system can also include a support structure for one or more components of the imaging system. The support structure can include an upright post, and an upper arm which can extend from an upper portion of the upright post, and optionally a lower arm extending from the upright post. The upper arm and any lower arm articulate independently. The support structure can be a fixed support, including a wall or other building or vehicle structural element. The support structure can also be a mobile support.

The present invention also provides an imaging system for real-time visualization of sub-surface structures in a body part of a mammal, the system including: a near-infrared (nIR) illumination source that emits nIR light that trans-illuminates the body part; a camera that captures the trans-illuminating nIR light, the camera optionally including a zoom lens to provide a detection field of view at a long working distance for the camera from the body part, the long working distance being sufficient to avoid the camera obstructing a visual field of view of the medical personnel when performing a medical procedure on the body part; a targeting system associated with the camera for indicating a focus location of the zoom lens and a center of detection field of view; an image processor for converting the captured trans-illuminating nIR light to an image signal; a visual display device attached to a distal end of the lower articulating arm and including a visual display screen; and at least one controller for sending a control signal to the camera, for sending power and control signals to the nIR illumination source, and for transmitting the processed image signal to the visual display screen.

In another aspect of the invention, the nIR illumination source is a disposable nIR light source device comprising a nIR-emitting light emitting diode (nIR-LED).

An imaging system of the invention can also include a filter for passing nIR light within a passband between 700 nm and 1000 nm.

In another aspect of the invention, the camera further includes an imaging processor that provides a logarithmic response to the intensity of nIR light detected, and a 16-bit gray scale resolution. In a further aspect, the controller can include a computer, wherein the image processor is integral with the camera or the computer, and wherein the image processor provides a logarithmic response to the intensity of nIR light detected, and a 16-bit gray scale resolution.

In another aspect of the imaging system of the invention, the first arm and the second arm are independently swivelable on the upright post.

In another aspect of the imaging system of the invention, the visual display device is a touch-screen, display-integrated computer.

In another aspect of the imaging system of the invention, the controller pulses and/or adjusts the intensity of the illumination output of the nIR illumination source, and controls a gate opening in the camera for capturing temporal image signals in synchronization with the pulsed maxima of the nIR illumination source output.

The present invention also provides an imaging system for real-time visualization of sub-surface structures in a body part of a mammal, the system including: a near-infrared (nIR) illumination source that emits nIR light that trans-illuminates a mammalian body part; a camera including a zoom lens to provide a detection field of view at a long working distance for the camera from the body part, the long working distance being sufficient to avoid the camera obstructing a visual field of view of a medical personnel when performing a procedure on the body part; a targeting system for indicating a focus location of the zoom lens and a center of detection field of view; an image processor for converting the captured trans-illuminating nIR light to an image signal; and a visual display device including at least one controller for sending a control signal to the camera, and for sending power and control signals to the nIR illumination source, and a display screen that receives and displays the processed image signal.

Another aspect of the present invention is a method for visualizing of sub-surface, including sub-dermal, structures in a body part or extremity of an animal, including of a mammal, comprising the steps of: positioning a camera disposed to capture images of a procedure on the body part; manipulating the camera to a field of view detecting position by aiming a targeting system at the body part to establish a center of detection field of view, and adjusting the focus location of the zoom lens; attaching a nIR illumination source for fixed positioning to an under-surface of the body part, and powering the nIR illumination source to trans-illuminate the body part; manipulating a viewing screen to a viewing position in the visual field of view of the user when performing the procedure; detecting the real-time trans-illuminating nIR light into a real-time trans-illuminated image; and viewing the real-time trans-illuminated image of the body part on the viewing screen while performing the procedure on the body part.

In another aspect of the invention, a further step includes manipulating the controller to change the detection field of view of the animal extremity by adjusting the zoom of the camera. The zoom feature can also increase the image size of the view field, enabling close-up or magnified views of the procedure field.

Another aspect of the present invention is a method for real-time visualization of sub-dermal body structures in a body part of a mammal, comprising the steps of: providing an imaging system according to the invention; positioning the camera can be above the eye-line (level of the eyes) of a medical personnel when positioned to perform a medical procedure on an extremity of a mammal, to avoid obstructing a visual field of view of the medical personnel; manipulating the camera attached to the distal end of the upper arm to a field of view detecting position by aiming a targeting system at the body part to establish a center of detection field of view, and adjusting the focus location of the zoom lens; attaching the nIR illumination source for fixed positioning to an under-surface of the body part, and powering the nIR illumination source to trans-illuminate the body part; manipulating the viewing screen attached to the distal end of the lower arm to a viewing position in the visual field of view of a personnel when performing the procedure, typically a medical procedure; detecting the real-time trans-illuminating nIR light into a real-time trans-illuminated image; and viewing the real-time trans-illuminated image of the body part on the viewing screen while performing the medical procedure on the body part.

Another aspect of the invention is a multi-functional control feature in a nIR trans-illumination and imaging system that includes a nIR light emitting source, a camera for capturing trans-illuminating nIR light through a body portion of a patient, a visual display device for displaying a trans-illuminated image of the body portion, and a computer for controlling the nIR light emitting source, the camera, and the visual display device, and for optionally further processing of the captured image and displaying the processed image on the visual display device. The computer can include a single-action, multi-functional control feature, or a multi-action, multi-functional control feature. The multiple functions of the control feature include the emission intensity of the light source, and at least one of the following image processing features: camera gain, sharpness, and camera spatial resolution.

The multi-functional control feature can be positioned between a first position associated with a first imaging condition that employs low light emission from the nIR light source, and at least one of low camera gain, and high camera spatial resolution, and high image sharpness, and a second position associated with a second imaging condition that employs high light emission from the nIR light source, and at least one of high camera gain, low camera spatial resolution, and low image sharpness.

A multi-action, multi-functional control feature provides at least two control features that operate between a first position and a second position. The pair of control features can be operated, or can operate, independently, or optionally they interactively can be selectively locked or linked together to operate together. One of the controller provides control of the nIR light source intensity while the other controls nIR sensitivity and image resolution. The nIR Sensitivity and the nIR illumination intensity are selected and optimized to obtain optimal visual images.

A single-action, multi-functional control feature operates between a first position and a second position. The first position is associated with a first imaging condition that employs low light emission from the nIR light source, low camera gain, and high camera spatial resolution, and high image sharpness, and is typified by the imaging of neonate patients. The second position is associated with a second imaging condition that employs high light emission from the nIR light source, high camera gain, low camera spatial resolution, and low image sharpness, and is typified by the imaging of adult patients with large muscular body parts. Operating the control feature at and between the first and second positions provides simultaneous and interconnected control of both the nIR light transmission and camera and processor setting between the two extremes.

Another aspect of the invention is a method for real-time visualization of sub-dermal body structures in a body part of an animal, comprising the steps of: a. providing a system including a nIR light emitting source, a camera for capturing trans-illuminating nIR light through a body portion of a patient, a display device for displaying a trans-illuminated image of a body portion of the patient, and a computer for controlling the nIR light emitting source, the camera, and the display device, and for processing of the captured image and displaying the processed image on the display device; providing a multi-functional control feature that operates the intensity of the light source and at least one of camera gain, camera spatial resolution, and image sharpness, the multi-functional control feature positionable between a first position associated with a first imaging condition that employs low light emission from the nIR light source, and at least one of low camera gain, and high camera spatial resolution, and high image sharpness, and a second position associated with a second imaging condition that employs high light emission from the nIR light source, and at least one of high camera gain, low camera spatial resolution, and low image sharpness; initiating an imaging procedure of the body portion of an animal patient; and selecting the position of the multi-functional control feature in accordance with the nIR transmission requirements of the body portion, to provide control, typically simultaneous and interconnected control, of the light emission from the nIR light source and the at least one of camera gain, camera spatial resolution, and image sharpness.

The devices, systems and methods are described for imaging the extremities and body parts of animals. The invention is particularly useful for imaging of mammals including humans, and also other genus and species of living creatures, including birds, fishes, amphibians, and reptiles, other vertebrates, and invertebrates, for various medical and biological applications, including by example, drugs testing.

### BRIEF DESCRIPTION OF THE FIGURES

FIG. 1 shows an illustration of an apparatus for imaging of nIR light trans-illuminating a patient's body part.

FIG. 2 shows an apparatus for imaging of nIR light trans-illuminating a patient's body part.

FIG. 3 shows a schematic diagram of the nIR light illumination and trans-illumination through the patient's extremity, and the power and control connections for the light source, camera, and visual display device.

FIG. 4 illustrates a visual display device showing a patient's hand and a touch screen interface with a single, multi-function slide mechanism for controlling the camera, the light source, and the image processing.

FIG. 5 illustrates another embodiment of a visual display device showing the patient's hand and a touch screen interface with a dual slide mechanism for controlling the camera, the light source, and the image processing.

### DETAILED DESCRIPTION OF THE INVENTION

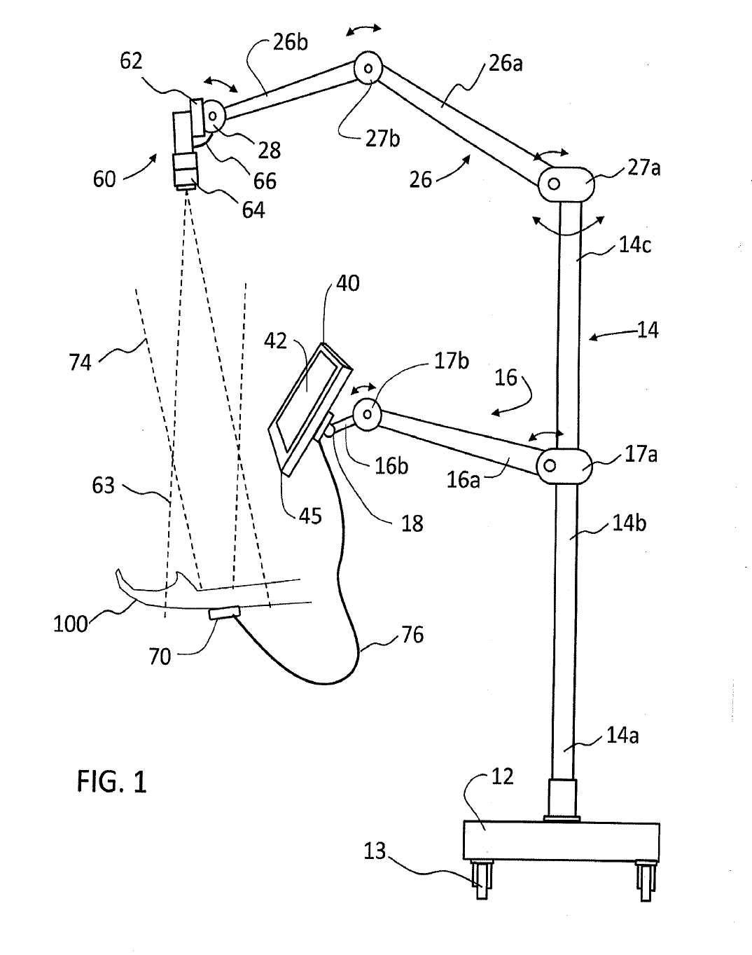

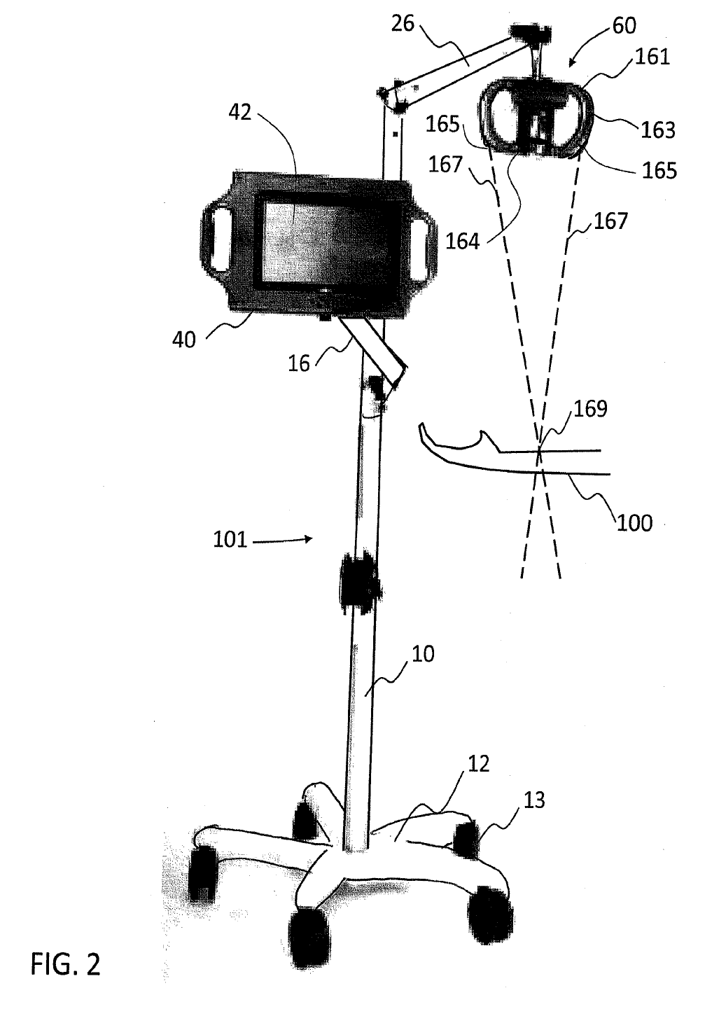

FIG. 1 shows an imaging system 1 for use by medical personnel for real-time visualization of sub-dermal body structures of an animal patient. A support structure is illustrated as a mobile stand 10 that provides a support structure for a camera 60 and a visual display device 40. The mobile stand 10 includes a base 12, an upright post 14 attached to the base 12 at a lower portion 14a. The post 14 includes an intermediate portion 14b, and can include an upper portion 14c. The base 12 extends laterally to provide adequate stable support for the upright post and its parts and accessories to prevent tipping. The base 12 is typically a weighted circular or rectangular platform of heavy material or having added weight for stability (as illustrated in FIG. 1), and may include a plurality of radially extending legs (as illustrated in FIG. 2) that ensure stability of the mobile stand and the entire system. Wheels 13 can be used on the base 12 for rolling the mobile stand 10 into position for the procedure, which can optionally be blocked or locked. The mobile stand is light weight and stable, with the post 14 and extending arms positioned at a height sufficient so as not to interfere with hospital beds and bed rails.

A lower arm 16 extends from the intermediate portion 14b of the upright post 14 and can be articulated into a position for optimal viewing of the visual display 42 of the display device 40 by the medical technician. A first lower arm segment 16a extends from a hinged connector 17a along the intermediate portion 14b. The hinged connector 17a can be fixed to the upright post 14. The hinged connector 17a may optionally include an adjustment mechanism so that the whole lower arm 16 assembly can be selectively moved upwardly and downwardly to a stationary position vertically along post 14. The first lower arm segment 16a can be configured to pivot selectively at the hinged connector 17a in a vertical plane (for example, out to 80° up or down from horizontal) or to swivel selectively in a horizontal plane (for example, out to 180° left or right) around the axis of the post, using well known joint means. A second lower arm segment 16b can be attached at a manipulable connector 17b to the distal end of the first lower arm segment 16a, for similar movement in the vertical and horizontal planes.

The display device 40 is attached to the distal end of the second lower arm segment 16b at a manipulable connector 18. The connectors 17a, 17b, and 18 can be configured for pivoting, swiveling or rotating along the axis by well-known means, for infinite positioning of the display device. The combination of connectors at 17a, 17b and 18 enables location of the display device according to the medical technician's personal preference to visualize the vascular image on the visual display for its use by the technician in performing a vascular access procedure while at the same time not obstructing the view of the patient, particularly the patient's face, as a means for the technician to continually assess the patient's condition and response to the procedure. Because of the ease of moving the visual display provided by connectors 17a, 17b and 18 and locking of the wheels 13 of the base 12, positioning of the visual display can be performed without disturbing the location of the mobile stand 10 or position of camera 60.

An upper arm 26 extends from the upper portion 14c of the upright post 14, above the lower arm 16, and can be articulated into a position for optimal capturing of the trans-illuminating nIR light 74 from the light source 70 by the camera 60. A first upper arm segment 26a extends from a hinged connector 27a at the distal end of or along the upper portion 14c. The hinged connector 27a can be fixed to the upright post 14. The hinged connector 27a may optionally include an adjustment mechanism so that the whole upper arm 26 assembly can be selectively moved upwardly and downwardly to a stationary position vertically along post 14. The upper arm 26 can be configured to pivot selectively at the hinged connector 27a in a vertical plane or to swivel selectively in a horizontal plane around an axis of the post, using well known joint means. A second upper arm segment 26b can be attached at a manipulable connector 27b to the distal end of the first upper arm segment 26a. The camera 60 is attached to the distal end of the second upper arm segment 26b at a manipulable connector 28. The connectors 27a, 27b and 28 can be configured for pivoting, swiveling or rotating along the axis by well known means, for infinite positioning of the camera.

The upper arm 26 extends from the upright post 14, and the upper arm members 26a and 26b have sufficient length that the camera can be positioned across a hospital bed, table or gurney above a patient body part and at a sufficient height over the body part to provide the typical camera working distance of 12 to 36 inches, to enable positioning the camera 60 near or above the level of the eyes of the medical practitioner who is positioned to perform the medical procedure. This positioning of the camera further prevents its obstructing the medical personnel's visual field of view of the image area of the procedure when performing the medical procedure on the body portion. The length of the upper arm members 26a and 26b also permit positioning of the camera into and over a variety of medical-related equipment and facilities, including over a neonatal infant incubator at the typical working distance so that a procedure can be performed on the infant without removing the infant from the incubator, in critical care facilities, and over an operating room table.

The lower arm 16 and the upper arm 26 are constructed of aluminum, steel, or high strength plastic tubular members for strength, light weight, and passage of electrical and control wiring between the electronic components of the system. The joints and connections between sections of the arms and between the arms and the devices can include, for example and without limitation, springs, friction-based adjustments, tensioning joints, weight balancing means, and quick-release fasteners to provide adjustable and stationary positioning for independent pivoting or swiveling of the arm members and the devices.

Alternatively, a support structure from which at least one of the upper and lower arms can depend can be a fixture. Non-limiting examples of fixtures to which the support structure can be fixed include a table, or bed, and a wall, and the fixture can be a portion or element of a building, field hospital, water vessel, or air-based emergency vehicles such as ambulances and helicopters. BTW, these do not vary too much from the figures that you show except for the mobile stand. such as

The base 62 of the camera 60 is attached to the distal end of the upper arm 26 at the manipulable connector 28 by well known means. The camera 60 can be articulated into position for optimal viewing of the nIR light reflecting or trans-illuminating the body portion during the procedure.

To obtain a detailed image and a full field of view of the body portion, with the camera positioned up and away from the work area of the medical personnel performing the procedure, the camera 60 can employ a zoom imaging feature. The zoom feature can include a zoom lens 64, as illustrated in FIG. 1, or digital zoom processing, alone or in combination with a zoom lens. The zoom lens 64 can be a fixed zoom lens selected to provide a fixed field of image at a selected, predetermined distance from the body portion, or can have a variable zoom feature that is either manually adjustable or remotely adjustable electronically from a controller. The zoom lens system may include a field of view capability with a broad range ratio of object size to image size. The range ratio can include 1:2 to 5:1, or more, including a 1:1 “life size” ratio. A smaller field of view provides a magnified image that facilitates close-ups and an increased size of view for neonate and pediatric patients. At the same time, the lens system may be configured so that the object lens distance remains the same and remains in focus. An autofocusing capability may be included in the camera 60 selected for use in system 1. The long working distance of the camera provided by the zoom lens is sufficient to avoid the camera obstructing a visual field of view of the medical personnel when performing a medical procedure on the patient. The typical working distance from the lens of the camera to the object (the body portion of the patient) is 4-36 inches, with examples of a more typical working distance being about 4-26 inches, 12-36 inches, and 22-24 inches.

The camera 60 is typically a solid-state, digital nIR-sensitive camera. A non-limiting example of a solid-state, digital nIR-sensitive camera is a Sony ICX618AQA, having an interline CCD solid-state image sensor 69 with a square pixel array which supports VGA format. The Sony ICX618AQA includes progressive scan that enables all pixel signals to be output separately within approximately 1/60 second, and employs the EXview HAD CCD™ that includes near-infrared light region typically in the range of from about 700 nm to about 1000 nm, as a basic structure of HAD (Hole-Accumulation Diode) sensor.

A narrow bandpass filter 68 can be used to pass near-infrared light of a selected range, typically between 840 nm and 875 nm, and more typically about 850 nm+20 nm. An electronic interface on the camera sends an image signal and other data concerning camera operation to a controller, and sends power and control signaling from the electronic interface to the camera. Other systems accomplishing the intended purpose may be selected by one with skill in the art within the intended scope of the teachings herein and of the appended claims.

The system provides independent positioning of the camera and the display device, such that moving the viewing screen out of the way temporarily or adjustment of the viewing screen during use does not require re-manipulating and positioning of the camera. This saves substantial time for the medical personnel and reduces the risks of making an error in, or overlooking some aspect of, the medical procedure.

FIG. 2 shows another imaging system 101 for real-time visualization of sub-dermal body structures of a patient, including a mobile stand 10 that provides a support structure for a camera 60 suspended from an upper arm 26 and a visual display device 40 suspended from a lower arm 16, with a base 12 having five radially extending legs with casters 13 for stable mobility. The castors 13 can include a lock to limit rolling movement of the stand 10. The camera 60 is mounted on a bracket 161 having a pair of extending handles 163 to aid positioning and aiming of the camera.

To aid in the determination of the camera focal distance and an optimal image focus, and for directing the field of view of the camera at the body portion target, the imaging system can employ a targeting system. A targeting system can comprise a convergent laser spotting device can include intersecting light (e.g., using laser diode lights or incoherent LED light sources) to generate two points of light that converge at a point of convergence at a focal range or distance from the camera lens. In one embodiment, the point of convergence of the targeting system is a distance (the convergence point distance) within the intended camera operating zone of 12-36 inches; for example, 22 inches. The convergent laser spotting device or mechanism indicates a reference distance of the camera, projected toward the body part, and a center of detection field of view of the camera image. Typically the targeting system works at least within the camera operating zone of 12-36 inches. An example of a laser focal distance system is described in Laser Ranging: a critical review of usual techniques for distance measurement, Markus-Christian et al., Optical Engineering, Vol. 40, No. 1, p. 10-19, 2001, the disclosure of which is incorporated herein by reference.

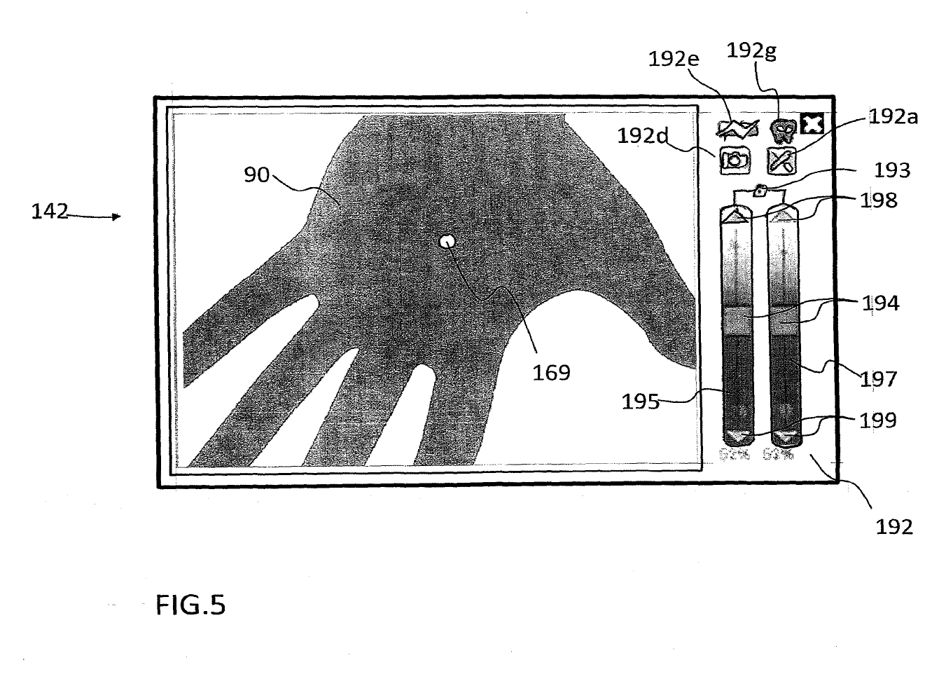

FIG. 2 shows the location of a pair of laser pointers 165 on the underside of the bracket 161 of the camera unit. The laser pointers orient the camera with respect to the area of the body part to be imaged. The two laser pointers emit beams of light (typically red light beams) along a beam path 167 to intersect at a fixed-distance, single intersection point 169. The two laser pointers 165 can be powered ‘on’ by a dedicated power switch, or by the computer that controls the power and control to the camera. If the surface of the body part (for example, the skin of the forearm of a patient) is positioned at the point of convergence of the targeting system, then a single visible point of light appears on the surface. If the body part is positioned closer to the lens, or farther from the lens, than the convergence point distance, then two points of light will appear a converging distance apart on the surface, proportional to the distance of the surface from the convergence point distance. Typically within the intended camera operating zone of 12-36 inches, either or both points of light appear on the surface. The location of the beam paths 167, and their intersection point 169, can be observed as visible points of light on the surface of the patient's body, and on the visual image presented on the visual display screen, as illustrated in FIG. 5

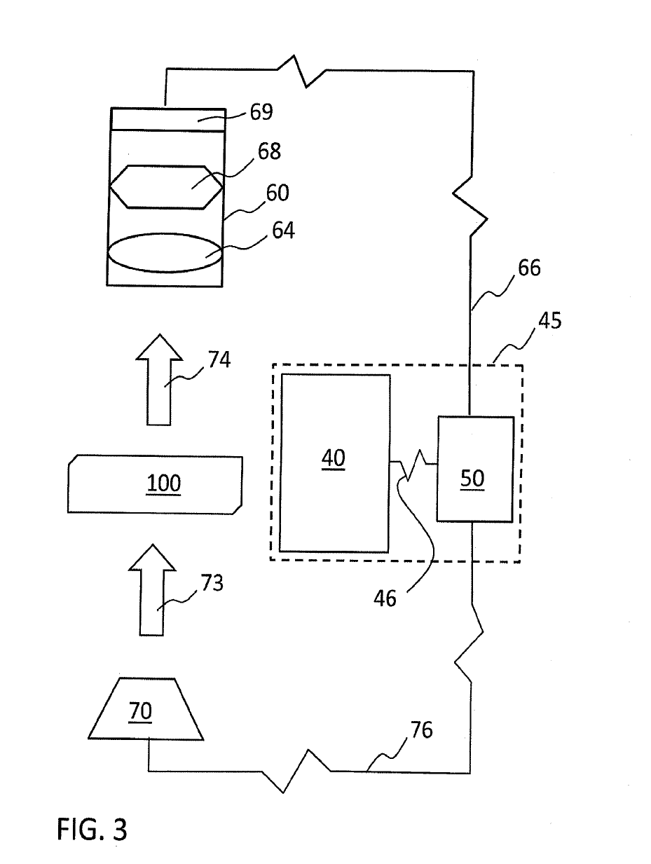

A lever 164 on the bracket 161 can be manipulated within a slot that is labeled with a scale of magnification factors from about 1× to about 2.25×. The lens can be a macro zoom lens that allows image zoom without object distance adjustment, which means that once the image through the lens is in focus, the image remains in focus through the zoom range. In this way the 22 inch distance and center of the field of view indicated by the convergent laser beams is consistently true even as the lever 164 is adjusted to zoom in on pediatric subjects for a magnified view. As shown in FIG. 3, emitting nIR light 73 is provided by a nIR light source 70 that is attached in photo-communication with the body portion (extremity) 100 of the patient.

The nIR light source is preferably small, disposable light source that is attachable to the skin surface of the patient so that the nIR light passes directly into and through the body portion, and is securable to the body portion to avoid movement or jostling of the light source during use. Examples of lights sources for emitting nIR light for imaging include coherent laser diodes and non-coherent light emitting diodes (LEDs). The LED typically emits nIR light in the range of 700 nm to 1000 nm. Preferred is an LED with an emission 73 within the range of 810 nm to 880 nm. The disposable light source (hereinafter, DLS) can have a plastic release liner on the light-emitting surface that allows the medical personnel to survey the body portion for veins and arteries, for example, for the best place to perform the vascular access procedure without exposing and disrupting an adhesive hydrogel. Once the desired position has been determined, the release liner can be removed (peeled off) from the hydrogel adhesive base material that provides both gentle adhesion to the skin (i.e., for neonates, pediatrics, and geriatrics) and optical coupling of the nIR illuminator (typically a nIR-LED) and the skin of the patient. The DLS provides for hands-free use during the procedure, while its single use nature serves as a barrier to spread of disease. The DLS can include one, two, three, four or more light emitters, depending upon the portion of the body to be imaged and the requirements of the medical procedure being viewed. The DLS can also have a proximity sensor that controls current to the nIR emitting diode, allowing the light source to turn on only when the DLS is in proximate contact with the patient's skin. An electronic interface is connected to the nIR illumination source for receiving power (in cases where the light source does not have on-board battery power) and for control signals. The electronic interface can be a wired interface that connects the light source to a remote controller, or can be a wireless interface, including an optical or radio frequency signal.

In an embodiment of the invention, the nIR illumination source is a single use or disposable light source (DLS) device that includes a light-directing and transmitting structure that can be applied to the skin surface of a portion of the body and a light source supported by the structure, including, but not necessarily limited to, a near-infrared light source. The device provides illumination of a body portion, and is useful in conjunction with systems and methods for real-time non-invasive visualization and identification of veins, arteries and other subcutaneous structures and objects in the body, in the administration of medical treatment to a patient, including facilitating intravenous insertion or extraction of fluids, medication or the like, and various surgical and diagnostic procedures affecting veins and arteries. The illumination can include trans-illumination, reflection, side illumination and backscattering. In addition, this light source permits the detection and identification of other natural subcutaneous structures and foreign objects such as metallic or plastic objects such as needles, stents, catheters, or fiber optic devices, or other non-natural items that could be present as a result of an accident or placed in situ for prosthetic purposes, or for the administration of medication or other infused substances.

The DLS can also include a proximity sensor for detecting when the DLS is positioned in proximity to the surface of the body portion of the patient. The proximity sensor controls the flow of current to the light source, and turns ‘on’ (delivers power to) the light source only when the light-emission pathway of the DLS is in close proximity to or in contact with the body portion, and which turns off the flow of current of the light source when the DLS is removed from proximity to the body portion. The proximity sensor significantly limits and preferably prevents light, especially near-infrared light, of the DLS from emitting generally in a direction other than the body portion, to avoid inadvertent light emissions that would become noise in the detected image or could enter the eyes of the patient, medical staff, or bystanders.

The DLS uses electrical power for the light source, and can include a layer or film of an electrically insulating material as a means for isolating electrically the light source, and any optional proximity sensor, from the body portion of the patient. The layer or layers of electrically insulating film or coating material prevents any electrical current flowing from or to the light source and associated electrical components of the DLS from flowing through the potentially electrically conductive conforming layer that is in direct contact with the skin of the body, thus avoiding and preventing electrical shocks or sensations or from interfering with additional medically placed instrumentation or sensors in the vicinity of the light source. In addition, the isolating layer also insulates the body surface from heat generated by the near-infrared light emitting diodes commonly used for illumination purposes associated with imaging the internal structures of the body.

The DLS can also include a light source wherein the source of electrical power and a controller for the light source are disposed remote from the DLS, to minimize the components, features, cost and complexity of the DLS. The simplicity of the design and components of the attachable and disposable light source can significantly reduce the cost of such device, allowing its use in a wider variety of medical procedures involving vascular access and subcutaneous imaging of the vasculature and the structures, or objects (endogenous or exogenous) with the body. The DLS can also include a disposable or replaceable light source, and a reusable structure that holds and electrically connects the light source and proximity sensor to a source of power and control. In addition, the DLS may be configured to be battery-powered via an on-board battery, and may be directly wired for power to an external device, including the display device 40 or other source of power.

A description of a suitable nIR light source device and its means of powering and control are described in U.S. Pat. No. 7,925,332, issued to Crane, supra, in U.S. Provisional Patent Application 61/513,689, filed Aug. 1, 2011, entitled “Disposable Light Source for Enhanced Visualization of Subcutaneous Structures”, and International Application PCT/US2012/49231, filed Aug. 1, 2012, the disclosures of which are incorporated herein by reference.

An important issue in the trans-illumination imaging of body portions with nIR light is the wide range of light intensities that need to be transmitted through different human body extremity types and conditions. For example, neonate's and children's hands are relatively thin, and will allow passage of a higher light transmission than, for example, the forearm of an adult male, which is much thicker. It is estimated that the difference in transmission between various body portion types is in some instances at least four orders of magnitude (10,000×) or more. To provide effective imaging across such a wide variation in light intensity, the captured image processor can employ a logarithmic response to light irradiance and 16 bits of intensity resolution.

Image processing can be performed on a computing device 50 remote from the display device 40, or can be performed within or on the display device 40 with an integrated computer 50. The computer 50 can be interfaced wirelessly or with a wired connection via communication path 46 with the display device 40, and/or interfaced wirelessly with a wired connection via communication path 66 with the camera 60, and/or interfaced wirelessly or with a wired connection via communication path 76 with the light source 70. The computer 50 and the display device 40 can be fixed to the system 1, or either or both can be portably carried by the medical technician. The display device 40 can include a computer 45 with an integrated visual display screen 42 that allows the technician to control each of the nIR light source operation, the camera operation, and the captured image processing directly from the display-integrated computer, using on-screen tables, menus, and manipulation of the controls for the devices. The display-integrated computer 45 is operatively connected to light source 70, directly through lead wired or wireless communication path 76 using well known wireless communication devices and methods. The display device 40 can also include a view display with dedicated permanent or semi-permanent processing and data-storage memory. The display can include liquid crystal displays (LCDs), and others. The size of the display can be selected to meet the requirements of the technician and for the medical procedure being accessed The size of the display can range from about 15 inches or more, to between about 7 to about 15 inches, and to as small as about 2 inches to about 7 inches.

The image signal can include a monochrome, gray-scale image signal that varies the shade of gray based on the intensity of the nIR light received. The processed image signal can be displayed for viewing in a gray or in a hue of any other desired color.

The display screen 42 can include a touchscreen that that can detect the presence and location of a touch within the display area. The resulting displayed image on a touchscreen display 42 can be selectively sized by the medical personnel or user to suit the need, for example, using the thumb and index finger alone or in combination to “size” the field of view 63 (FIG. 1) of the camera output to a specific view of interest.

The resulting captured image can be processed and enhanced computationally, including by well known means. The display-integrated PC can also include programming for enhancing the processed image of the nIR light, to highlight specific anatomical features or tissue types.

A visual display device presents an image of the trans-illuminated body portion for unaided viewing by the technician. The visual display device can be a stand-alone unit that provides only the visual display screen, or can include the visual display screen integrated with one or more computing and control devices. In the embodiment illustrated in FIG. 1, the flat-panel touch screen 42 of the display-integrated computer 40 (FIG. 1) provides an image that can be large, typically of 12-inch or smaller in diagonal, and of high resolution, with a minimum of 800×480 pixels per inch, and typically 1280×720 to 1280×1024 pixels, that enables the area of the procedure on the body portion to appear “life size” on the visual display screen 42.

Operation and control of the nIR illumination source, the camera, and the imaging and the display functions are performed on a computer, and can include, but not be limited to, programming for touch control of the screen image size (for example, between full screen and partial screen images), selection of visualized image color (for example, gray or green), for capturing and displaying still-photo or video images, for on-board archiving, and for image processing including attenuation of brightness, contrast and saturation of the processed image from the camera.

The computer can be a commercially available computer with an operating system that can run commercially available software applications to perform the various operations of the system described herein, The computer can also operate on a proprietary operating system and with proprietary software that provides function to the camera, light source, and display, as well other functionality including, but not limited to, the image processing and enhancement, image and data archiving, and image and data live streaming to or over a local or public network.

A human interface with the computer can employ any of the well known means available, including wired or wireless keyboard, mouse or cursor positioning device, or a human finger(s) or capacitive stylus (on a touchscreen). A non-limiting example of a human interface is a graphic user interface (GUI) that allows the users to interact with the electronic components of the system using images rather than text commands. A GUI that employs a touch screen display device permits the user to use their finger(s) or a stylus to point at and touch the graphic images themselves to perform the control actions. The touch-screen interface can provide, for example, selection of menus and control features for the camera and the light source devices, for manipulation and storage of the captured image, and for transmission, storage and display of the manipulated and processed image to the visual display device. The touches by a user on the touch screen can include points with one or more fingers or a capacitive stylus, swipes across the surface of the screen, and pinches and expansions with two or more fingers in contact with the surface of the touchscreen.

The display-integrated computer 45 can be programmed to provide different rates of pixel binning that allow the technician to select from among, for example, high, medium and low resolution settings. The display-integrated computer includes menus that are accessible with a screen touch for data entry via an integral virtual keyboard, image and data manipulation, device selection and control, and power and battery-charge status. Data and images captured on the display-integrated computer can be exported using standard medical device data transmission language (i.e., DICOM) via USB (universal serial bus) port, Ethernet and/or a wireless network connection.

In an embodiment of the invention, the controller can be manipulated through the touch screen interface to provide integrated control of the emitting intensity of the light source, and one or more image data processing functions, including bin setting, gain, and sharpening. There is generally a need to image over a 10,000× or greater light intensity range.

In a first imaging condition, typified by neonate vascular imaging, the small and highly transparent anatomy of a neonate patient results in very high optical transmission of nIR light. The vessels are correspondingly small in size with fine details, and require high spatial resolution and optimal definition of vessels for viewing. The settings for processing the captured image under this extreme condition include low camera gain, low nIR light emission intensity, and high camera spatial resolution, and high image sharpness.

In a second imaging condition at the other extreme, typified by vascular imaging in an adult male, nIR transmission through the body part is very low due to the thick musculature of adult anatomy. In the adult, the vasculature is correspondingly large, such that a lower spatial resolution is needed for adequate viewing. This setting would require a maximum nIR light transmission for maximal transmission through the body part, along with high camera gain, low(er) camera spatial resolution, and low(er) image sharpness.

The camera spatial resolution is controlled by pixel binning Camera binning can be none (1×1), 2×2, 3×3, or 4×4. Pixel binning results in proportionally higher light sensitivity (2×2 binning would increase light sensitivity by 4×, 3×3 binning by 9×, and 4×4 binning by 16×) but with a corresponding lower spatial resolution. Pixel binning adds (sums) the values of the block of pixels defined by the bin size to create a single new pixel. Pixel binning is only practical when a high spatial resolution camera is used as all binning results in decreased spatial resolution. Image sharpness is a common image processing algorithm that amplifies a light to dark or dark to light adjacent pixel transition in effect increasing edge sharpness. This technique works well except when the gain of the camera is set high. With high camera gain the image sharpness function amplifies the noise present in high gain images resulting in an even lower signal to noise ratio noisier and therefore degraded image.

There are a wide variety of touchscreen-enabled graphic user interfaces (GUI) can be designed to perform any particular operation or function of the system, and may be limited only by the imagination of the GUI designer.

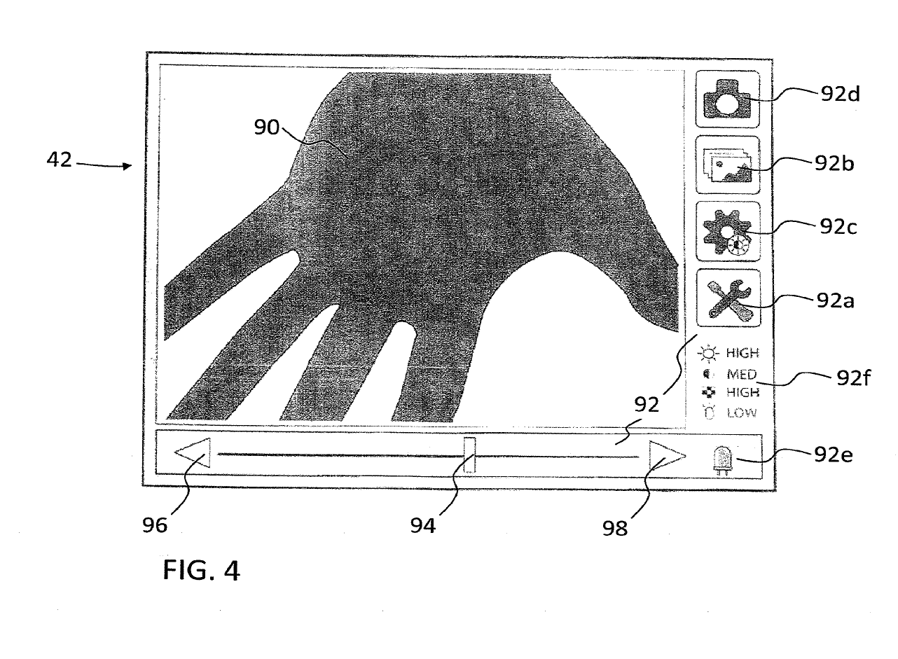

In one embodiment, the interface includes a GUI including an on-screen, single-action multifunctional slider as a control feature under the user/operator's control. A virtual sliding switch in an application running on the touch screen can be moved along a continuum between two ends of the slider, for operation of the light source between the two extreme imaging conditions. The virtual “sensitivity” slider adjusts the properties of the light source (nIR light intensity) and the camera (gain, sharpness, and pixel binning) at a predetermined combination of the settings along the range between minimum intensity and maximum intensity. Consequently, the low-transmission, high-sensitivity end of the virtual slider might be optimized for the neonate imaging extreme, while the high-transmission, low-sensitivity end might be optimized for the male adult muscular extremity. FIG. 4 illustrates a visual display screen 42 of the showing a patient's hand image 90 and a touch screen interface 92 as the on-screen graphic user interface (GUI) for controlling the camera 60, the light source 70, and the image processing of the computing device 50. The GUI 92 can include individual touch areas for various functions of the camera, light source and image processing. A single-action virtual slider 94 operates between the neonate imaging extreme end 96 and the adult forearm imaging extreme end 98. User-interface areas include a tools area 92a, a brightness area 92b, a contrast area 92c, a “save image” area 92d, a battery status indicator 92e, and a condition status area 92f.

The transition of the sensitivity slider from low to high effects the following image adjustments:

A) The drive current to the nIR trans-illumination light source (e.g., LED) proportionally adjusts from 1 ma at the low end to 80 ma at the high end, with a smooth transition there between.

B) The camera gain proportionally adjusts from 6 dB (2×) at the low end to 41 dB (112×) at the high end.

C) The pixel binning changes from 2×2 at the low third of the sensitivity adjustment to 3×3 at the center third and 4×4 at the high end third of the adjustment. When a transition in binning size occurs there is a corresponding change in sensitivity (2.25× at the first transition and 1.78× at the second transition). To make this sensitivity adjustment seamless (smooth with no sudden changes in apparent sensitivity), when a binning transition occurs the camera gain will be corresponding decreased (−2.25× at the first transition and −1.78× at the second transition), to create a smooth seamless adjustment in image sensitivity.

D) The sharpness adjustment will also be utilized in a 3-step manner. The degree of sharpness enhancement can be classified as 0 (no sharpness enhancement), 1 (medium sharpness enhancement) and 2 (high sharpness enhancement). The sharpness effect will be set to 2 at the low third of the sensitivity adjustment, changed to 1 for the middle third of the adjustment, and dropped to 0 for the high-end third of the adjustment.

The result is a single adjustment feature that provides optimal viewing of extreme anatomical viewing requirements by simultaneous and interconnected control of both light transmission and camera sensitivity between the two extremes.

In another embodiment, the interface includes an on-screen graphic user interface (GUI) including an on-screen, dual slider as a control feature under the user/operator's control. FIG. 5 illustrates a display screen 142 showing a patient's hand image 90 and a touch screen interface 192 as the on-screen graphic user interface (GUI) for controlling the camera 60, the light source 70, and the image processing of the computing device 50. The GUI 192 can include individual touch areas for various functions of the camera, light source and image processing. A pair of vertical virtual slide controllers (sliders) 195 and 197 along the right hand side of the display provide control and adjustment for the separate functions of nIR sensitivity and resolution (195), and nIR light source intensity (197). User-interface areas include a tools area 192a, a “save image” area 192d, a battery status indicator 192e, and a DLS proximate status area 192g.

The two vertical sliders 195,197 permit the control of the levels of nIR sensitivity and the amount of nIR for effective imaging of different sized patients as well as different tissue thicknesses in individual patients. The architecture of the imaging chip used in a camera typically provides the highest level of nIR sensitivity with the least image resolution. The moveable slider bar 194 on each of the slider bars 195 and 197 can be moved up or down from 0 to 100% of function by touch or stylus, to increase or decrease the relative amount of nIR sensitivity (which is inversely related to image resolution) and nIR light intensity (the current provided to the DLS). The triangles 198,199 at the top and bottom respectively of each of the sliders 195,197 can also be used to move the slider bars 194.

A default condition interlocks the two slider bars 194, so that moving one slider bar causes an equivalent movement of the other slider bar. A lock icon 193 at the top of the sliders 195,197 indicates whether the slider bars 194 are locked together or are unlocked to permit independent movement. The slide bars 194 can be unlocked, and then locked again, by touching the lock icon 193 with a finger or a capacitive stylus. The independent movement of the slider bar for the nIR sensitivity and resolution slider 195 and nIR light source intensity slider 197 enables a user, with just a little experience, to adjust the two control settings to optimize imaging. The nIR Sensitivity slider bar adjusts the nIR sensitivity and image resolution. Image resolution is inversely related to nIR sensitivity. The maximum nIR sensitivity (100%) which might be needed for imaging through thicker tissue sections will provide the lowest image resolution. Image resolution can be increased by moving the nIR slider bar down, but at the expense of decreased nIR Sensitivity. The nIR Sensitivity must be balanced with the amount of nIR from the DLS in order to obtain optimal images of the vasculature. The amount of nIR is adjusted to provide an optimum amount of nIR to obtain good vascular and tissue images. Too much nIR illumination can “wash out” the image (overpower the image with light), so no or very poor images of vasculature are seen. The “washing out” of the image appears to glow white (or lighter) rather than showing a contrast image of vessels or tissue. Too little nIR (or too little nIR sensitivity) will result in a dark image with reduced clarity of the vasculature or no vasculature showing. In general, less nIR light intensity is needed with higher levels of nIR sensitivity.

After the controller settings have been made and the imaging system is ready for imaging of the procedure, the user can touch image portion of the touchscreen display with a finger or stylus, causing the image portion of the display to expand and fill the entire viewing area of the visual display, which hides the various control icons and sliders of the GUI. The expansion of the viewed image to full display increases the image magnification by approximately 0.5×. As a result, for example, the full-display magnification at the 1.5× setting of the zoom control lever actually increases to 2.0×. Touching the display a second time by the finger or stylus restores the partial screen image of vasculature, and restores the GUI with its various controls.

Processed images of vasculature that appear on the display can be saved for later downloading by touching the camera icon 192d with the finger or stylus. Downloading of the image to an external memory source can be done via an outlet communication means, (for example, a wired ports including a Universal Serial Bus (USB) port, or wireless transmission) that can be located on or within the display-integrated computer 45. The image storage file identity can be automatically assigned a number or replaced by some other file designation chosen by the user using a menu that appears on the GUI. The user's notes regarding the saved image can be entered with the image file via a virtual keyboard accessed in the menu.

The “tools” or “settings” icon 192a, shown is located just above the slider 197, opens an on-screen menu when selected, to modify and update the features of the system, including factory defaults and manual override of default settings. These features include the file saving function, image brightness settings, gamma (a complex function developed to compensate for the difference of human visual perception and digital image presentation), contrast, and image storage path. An example of a display-integrated computer with a touchscreen can include the IPad™ (Apple) which operates on a proprietary operating system, or an HP Compaq Tablet, a Blackberry Slate (RIM), and a Motorola Zoom, all of which operate with a Microsoft (Windows 7, Windows 8) operating system. The typical tablet-type computer has an instant-on solid-state hard drive, a graphical processor unit (GPU) and a central processor unit (CPU) and storage memory, enabling the display-integrated computer to be configured for controlling the operation of the light source and the camera, for adjusting and controlling image processing, and for editing, storing, displaying and transmitting nIR images.

The display-integrated computer 40 includes programming and control modules controlling the light source (DLS) 70, and the camera 60 and its electronic and mechanical components. In one aspect of the invention, the DLS includes a nIR-emitting mid-range LED, or plurality of LEDs. Optionally, the LED(s) is pulsed from ‘on’ to ‘off’ to provide nIR illumination during discrete temporal periods. The optional pulsing of the LED(s) from ‘on’ to ‘off’ can minimize the power consumed by the LED and reduce the heat generated by the LED. Pulsing the LED also allows for an increase in emission peak height which can increase the signal-to-noise ratio. The shutter openings can be gated with the pulsing of the LEDs so that nIR illumination occurs only during the time when the trans-illuminating light 74 is being captured by the camera, thus improving the signal to noise ratio.

Since the camera 60 is sensitive to both visible and nIR illumination, the display-integrated computer 45 also includes programming and control modules that detects the ambient light cycles, typically of fluorescent lighting (which is typical of the lighting found in hospitals and clinics), and synchronizes the nIR illumination with the minima of the ambient light cycle, as described in US Patent Publication 2004-0215081, published Oct. 28, 2004, entitled “Synchronization of Illumination Source and Sensor for Improved Visualization of Subcutaneous Structures”, the disclosure of which is incorporated by reference.

In a typical medical procedure, such as the insertion of a needle into the vein of a patient, the apparatus of the present invention produces an easy to interpret, X-ray-like planar image of the vasculature in the patient's arm, with a wide field of view. This result contrasts with images obtained by an ultrasound device, which produces cross-sectional images with a narrow field of view. The system is capable of providing high quality images of a wide variety of body portions, including, though not limited to, the forearms, wrists and hands of most adults, and including, though not limited to, the hand, wrist, forearm, elbow, upper arm, foot and ankle of an infant, as well as other anatomic portions of an infant that are not reliably imaged in adults. The type of medical procedures that will benefit from the use of the system include, but are not limited to, vascular access to arteries and veins for sampling, monitoring, intravenous administration of nutrients, fluids, electrolytes, and medications, trans-radial percutaneous coronary and vascular interventions, and contrast agent injection.

In a typically procedure for using the system 1 shown in FIG. 1, the display-integrated computer 45 is activated, and the digital nIR camera 60 is connected to the display-integrated computer 45 as described above and powered on. The technician positions the articulated upper arm 26 with the camera 60 mounted at its distal end to provide an image of the body part to be imaged with the camera approximately 22-24 inches above the patient's body part to be imaged. This distance is sufficiently long to place the camera out of the direct view, and the vicinity of the procedure, but is close enough with the zoom lens to provide a tight, detailed image field of the patient's body part. The technician adjusts the camera's zoom setting (optional) and focus using either manual controls, for example, levers (not shown), extending from the lens 64, or remote controls on a drop-down menu of the display-integrated computer 45, until a well-focused, tight image of the procedure site is obtained.

A disposable light source (DLS) device 70 is removed from its protective foil pouch, connected electrically to the display-integrated computer 40 via wired communicated path 76, and power and pulsing signal controls are activated to the DLS 70. A guide slot is placed over the input port on the computer 40 to assist connecting the wired connection of the DLS into the display-integrated computer 40. The DLS 70 can include a proximity sensor (described in International Application PCT/US2012/49231, filed Aug. 1, 2012, the disclosures of which is incorporated herein by reference) that prevents the delivery of power to illuminate the LED until the DLS is placed into proximal contact with the skin of the body part 100 of the patient. Prior to removal of the plastic film that covers the hydrogel-interface layer of the DLS, the DLS has been placed against the skin on the underside of the patient's wrist, hand or other body part to be imaged, which activates the pulsing of the nIR LEDs of the DLS. The medical technician surveys the wrist, hand or other body part to be imaged monitoring the nIR image of the wrist on the touch screen 42 of the display-integrated computer 45, until the desired location of placement of the DLS is identified. The technician then removes the plastic film to expose the hydrogel adhesive layer, and attaches the adhering DLS to the desired location on the underside of the wrist, hand or other body part to be imaged. During the procedure, the adhesion of the hydrogel to the skin is sufficient to hold the DLS in optically-coupling contact with the skin at its chosen position, and frees the hands of the operator or technician to perform other tasks. The DLS provides for hands-free operation during a vasculature access procedure.

Upon attaching the DLS to the skin, the proximity sensor is activated and power control is reestablished to the DLS. Using either manual levers or touch screen and drop-down menus on the display-integrated computer, the technician makes minor adjustments, as needed, to the focus of the lens 64 of camera 60, to the power output of the DLS, and to the brightness, attenuation, and contrast of the acquired image displayed on the touch screen. The display-integrated computer at the end of the lower arm is then articulated so that the touch screen is within easy reach and view of, yet out of way of the actions of, the medical personnel who performs the procedure.

The visual images that are transmitted to the visual display screen 42, including single shot images or a streaming video of the images, can be archived and stored on the display-integrated computer itself, or transmitted or re-transmitted to a remote storage and/or display device to provide real-time output or archive retrieval of images and data over a local or public network, and including of networked online storage where data is stored in virtualized pools of data storage that generally hosted in internet-based data centers by third parties, known as cloud storage, using either a wired connection or a wireless connection, including RF.

Visual images, including singles shots and video images, can be fixed in some permanent or semi-permanent form onto a data storage media (for example, a hard drive flash drive, or other), and identified by an identify (file name) and data storage address or location to enable later access by a user. The file name can be revised or renamed, and the identities of one or more data files can be archived, changed, or otherwise customized as needed or desired.

1. An imaging system for real-time visualization of sub-surface structures in a body part of a mammal, the system including:
1) a near-infrared (nIR) illumination source that emits nIR light that trans-illuminates the body part;
2) a support structure that includes an upright post, a lower arm extending from the upright post, and an upper arm extending from an upper portion of the upright post and including a distal end, wherein the upper arm and the lower arm articulate independently;
3) a camera attached to the distal end of the upper arm that captures the trans-illuminating nIR light, the camera including a zoom lens to provide a detection field of view at a long working distance for the camera from the body part, the long working distance being sufficient to avoid the camera obstructing a visual field of view of the medical personnel when performing a medical procedure on the body part;
4) a targeting system associated with the camera for indicating a focus location of the zoom lens and a center of detection field of view;
5) an image processor for converting the captured trans-illuminating nIR light to an image signal;
6) a visual display device attached to a distal end of the lower articulating arm and including a visual display screen; and
7) at least one controller for sending a control signal to the camera, for sending power and control signals to the nIR illumination source, and for transmitting the processed image signal to the visual display screen.

2. (canceled)

3. The system according to claim 1, wherein the nIR illumination source is a disposable nIR light source device comprising a nIR-emitting light emitting diode (nIR-LED).

4. The system according to any of claim 3, further including a filter for passing near infrared (nIR) light within a passband between 700 nm and 1000 nm.

5. The system according to claim 1, wherein the at least one controller includes a computer, wherein the image processor is integral with the camera or the computer, the visual display device is a touchscreen display-integrated computer, and the image processor provides a logarithmic response to the intensity of nIR light detected, and a 16-bit gray scale resolution.

6. A method for visualizing of sub-surface structures in a body part of a mammal, comprising the steps of:
a. positioning a camera disposed above the level of the eyes of a user, when positioned to perform a procedure on the body part, to avoid obstructing a visual field of view of the use;
b. manipulating a camera to a field of view detecting position by aiming a targeting system at the body part to establish a center of detection field of view, and adjusting the focus location of the zoom lens;
c. attaching a nIR illumination source for fixed positioning to an under-surface of the body part, and powering the nIR illumination source to trans-illuminate the body part;
d. manipulating a viewing screen to a viewing position in the visual field of view of the user when performing the procedure;
e. detecting the real-time trans-illuminating nIR light into a real-time trans-illuminated image; and
f. viewing the real-time trans-illuminated image of the body part on the viewing screen while performing the procedure on the body part.

7. (canceled)

8. The multi-functional control feature according to claim 10 wherein control is simultaneous and interconnected

9. The method according to claim 6, wherein the camera includes a zoom lens to provide a detection field of view at a long working distance from the body part, the method further including a step of adjusting the zoom lens.

10. A multi-functional control feature in a nIR trans-illumination and imaging system, the system including a nIR light emitting source, a camera for capturing trans-illuminating nIR light through a body portion of a patient, a visual display device for displaying a trans-illuminated image of the body portion, and a computer for controlling the nIR light emitting source, the camera, and the visual display device, and for optionally further processing of the captured image and displaying the processed image on the visual display device, the multi-functional control feature providing operation of the intensity of the light source and at least one of camera gain, camera spatial resolution, and image sharpness, the multi-functional control feature positionable between a first position associated with a first imaging condition that employs low light emission from the nIR light source, and at least one of low camera gain, and high camera spatial resolution, and high image sharpness, and a second position associated with a second imaging condition that employs high light emission from the nIR light source, and at least one of high.
